(19) United States
(12) Patent Application Publication
(10) Pub. No.: US 2026/0321037 A1
(43) Pub. Date: Oct. 15, 2026

(54) Machine Learning Models for Optimizing Cell Line Selections

(71) Applicant: AMGEN INC., Thousand Oaks, CA (US)

(72) Inventors: Yi Li, Van Nuys, CA (US); Yu Yuan, Irvine, CA (US); Ewelina Zasadzinska, Woodland Hills, CA (US); Ryan N. Kyger, Newbury Park, CA (US); Valerie Komatsu, Pico Rivera, CA (US); Becky Cheng, Thousand Oaks, CA (US); Elcin Icten Gencer, Medford, MA (US); Ju-Chun Huang, Revere, MA (US); William Heymann, Julich (DE); Austin Xiong, Somerville, MA (US); Shahab Golshan, Montreal (CA); Jack Albright, Brooklyn, NY (US); Erika Perez, Thousand Oaks, CA (US)

(21) Appl. No.: 19/642,085

(22) Filed: Apr. 8, 2026

Related U.S. Application Data
(60) Provisional application No. 63/786,256, filed on Apr. 9, 2025.

(51) Int. Cl. G06V 20/69; G06V 10/32; G06V 10/764; G06V 10/82; G16B 40/10; G16B 40/20

(52) U.S. Cl. CPC G06V 20/698; G06V 10/32; G06V 10/764; G06V 10/82; G16B 40/10; G16B 40/20

(57) ABSTRACT

A computational method of cell clone assessment comprises obtaining, by one or more processors and from a cell line development and assessment system, assessment data respectively associated with wells assessed by a cell line development and assessment system. At least a portion of the wells includes a candidate clone. The method further includes selecting a pair of candidate clones having a common parent cell line. The method includes predicting, for each pair of candidate clones, cell clone performance for the candidate clones by applying the assessment data as inputs to a machine learning model configured to predict a clone performance metric. The method further includes generating an output indicative of which candidate clone of the pair of candidate clones has a superior clone performance metric.

### CROSS-REFERENCE TO RELATED APPLICATIONS

This application claims priority to and the benefit of the filing date of provisional U.S. Patent Application No. 63/786,256 entitled “MACHINE LEARNING MODELS FOR OPTIMIZING CELL LINE SELECTIONS” filed on Apr. 9, 2025. The entire contents of the provisional application are hereby expressly incorporated herein by reference.

### FIELD OF DISCLOSURE

The present application relates generally to cell line (clone) selection techniques, and more specifically relates to techniques for predicting cell clone performance based on assessment data analyzed with a machine learning model.

### BACKGROUND

In the biopharmaceutical industry, large, complex molecules (e.g., proteins) known as biologics are derived from living systems. The general workflow for the development of a biologic begins with research and development. In this initial phase, a disease, or indication that represents an important unmet medical need is targeted. Researchers determine the potential drug candidates based on a proper target product profile, which govern aspects such as safety, efficacy, and route of administration, for example. Ultimately, through a combination of in vitro research and computational models, a specific molecule is chosen as the top drug candidate for the specific disease and target population.

FIG. 1 depicts a cell line development process 10. After the top candidate is selected, the blueprint for the molecule is formalized into a gene, and the gene of interest is inserted into an expression vector. The expression vector is then inserted into the host cell, in a process 11 known as transfection. The cell can incorporate the gene of interest into its own production mechanisms upon successful transfection, eventually gaining the ability to produce the desired pharmaceutical product.

Because each cell has unique characteristics, the product produced by each cell varies slightly, e.g., in terms of productivity (e.g., titer) and product quality. In general, it is more desirable to produce drugs with consistently high titers and consistently high quality, for reasons of economy and safety. High concentrations, or titers, of a product help to reduce the manufacturing footprint needed to generate desired production volumes and therefore save both capital and operating expenses. High product quality ensures that a greater proportion of the drug is safe, efficacious, and usable, which also saves costs. In the context of cell line development, product quality attributes are evaluated through assays conducted on the product of interest. These assays often include chromatographic analysis, which is used to determine attributes such as degree of glycosylation and other factors such as the proportion of unusable proteins due to truncations (clippings) or clumping (aggregates).

Based on criteria relating to productivity and product quality, the “best” cell line or clone is selected in a process 12 known as “cell line selection,” “clone selection,” or “clone screening.” The selected cell line/clone is used for the master cell bank, which serves as the homogeneous starting point for all future manufacturing (e.g., clinical and commercial). After transfection and selection, recovering cell pools at operation 14 allows cells to grow for a few days.

Ensuring a consistent product batch helps promote a more uniform and predictable pharmacokinetic and pharmacodynamic response in patients. If a “pool” of heterogeneous cells obtained after transfection is used to generate the product of interest, however, there may be many variants of the product generated. This is because during transfection, the gene of interest is integrated into candidate host cells in variable ways. For example, there may be differences in copy number (i.e., the number of integrated copies of the gene of interest) and other differentiating factors between the unique footprints of different cells. The manufacturing of the product of interest may also vary due to slight differences in the internal machinery of each individual cell, including the nature of post-translational modifications. These variations are undesirable, especially considering the need to ultimately control for and ensure a measured and safe response in the patient. Thus, it is typically required that the master cell bank cell line be “clonally derived,” in process 14, i.e., that the master cell bank only contain cells derived from a common, single cell ancestor. This theoretically helps ensure a large degree of homogeneity in the drug produced, despite slight, inevitable differences due to natural genetic variation through random mutation as cells divide. Therefore, the clone screening process is important in delivering not only a productive, high quality starting material, but also a singular cell line that complies with the “clonally derived” requirement. Cell clones can be imaged at operation 15, using processes such as microscopy with fluorescent proteins or reporter genes, or flow cytometry.

A clone screen process begins at operation 16. Clone screening can include a microtiter plate-based method of clone generation and growth, which may take two to three weeks. Hundreds of pooled, heterogeneous cells are sorted into single-cell cultures through processes such as fluorescence-activated cell sorting (FACS) or limiting dilution. After being allowed to recover to healthy and stable populations, these clonally-derived cells are analyzed, and select populations are transferred to a subsequent stage, where clone cells in small containers, such as spin tubes, 24-well plates, or 96-deep well plates are cultured in a “small-scale cell culture” (e.g., a 10-day fed batch process). In this small-scale process, boluses of nutrients are added periodically, and different measurements of cell growth and viability are obtained. Typically, hundreds or even thousands of these small-scale cultures are run in parallel. At the end of the culture (e.g., the tenth day), the cells are harvested for assays and analysis.

By analyzing the growth and productivity characteristics of the clones in the small-scale cultures, at stage 12, the “top” or “best” clones (e.g., the top four) are selected for scaled-up cultures that are run at a third stage 17. The scaled-up (or “large-scale”) process is useful because, relative to the small-scale cultures at stage 16, it better represents the process that will ultimately be used in clinical and commercial manufacturing. The scaled-up process may occur through a 15-day culture in 3 to 5 liter perfusion bioreactors, for example. These perfusion bioreactors accommodate more efficient transfer of waste and nutrients, thereby increasing overall productivity of the culture. Perfusion bioreactors are also typically associated with a higher number of measured variables, such as daily and continuous process conditions and metabolite concentrations, to enable tighter control and monitoring.

After the scaled-up process at stage 17, the media and product are collected and analyzed. Ultimately, at a fourth stage 16, the scaled-up run that yielded the highest titer and exhibited the best product quality attributes (PQA) is typically chosen as the “best,” or “winning,” clone. Finally, at a fifth stage 18, the winning clone is used as the master cell bank for characterization, and future clinical and commercial manufacturing use.

Conventional clone screening processes of the sort described above are extremely resource-intensive, typically taking several months and requiring hundreds or thousands of assays and cell cultures. As the pace of biotechnology quickens, however, and as an increased emphasis is placed on processing additional molecules in the early-stage pipeline, there is an increasing need for faster clone screening. Moreover, conventional clone screening processes lack standardized criteria for selecting which clones to advance to the next stage/bioprocess and, ultimately, selecting a winning clone, in part because the unique combination of modality, structure, and sequence characteristics for each different drug candidate means that different factors can be more or less important.

### SUMMARY

Embodiments described herein relate to systems and methods that create, evaluate, and/or apply predictive models of cell line and bioprocess performance in clone selection. In particular, robust machine learning models are created and used to reduce development timelines and resource usage while improving performance.

In one aspect, a method of cell clone assessment is provided. The method can include obtaining, by one or more processors and from a cell line development and assessment system, a plurality of assessment data respectively associated with a plurality of wells assessed by a cell line development and assessment system, wherein at least a portion of the plurality of wells include a candidate clone. The method can include selecting, by the one or more processors, a pair of candidate clones having a common parent cell line. Still further, the method can include predicting, by the one or more processors, for each pair of candidate clones, cell clone performance for the candidate clones by applying the plurality of assessment data as inputs to a machine learning model configured to predict a clone performance metric. The method can include generating, by the one or more processors, an output indicative of which candidate clone of the pair of candidate clones has a superior clone performance metric.

In additional aspects, a non-transitory computer-readable medium storing instructions for cell clone assessment is provided. The instructions can cause one or more processors to obtain, from a cell line development and assessment system, a plurality of assessment data respectively associated with a plurality of wells assessed by a cell line development and assessment system, wherein at least a portion of the plurality of wells include a candidate clone. The instructions can further cause the one or more processors to select a pair of candidate clones having a common parent cell line. The instructions can further cause the one or more processors to predict, for each pair of candidate clones, cell clone performance for the candidate clones by applying the plurality of assessment data as inputs to a machine learning model configured to predict a clone performance metric. Still further, the instructions can cause the one or more processors to generate, by the one or more processors, an output indicative of which candidate clone of the pair of candidate clones has a superior clone performance metric.

### BRIEF DESCRIPTION OF THE DRAWINGS

The skilled artisan will understand that the figures, described herein, are included for purposes of illustration and do not limit the present disclosure. The drawings are not necessarily to scale, and emphasis is instead placed upon illustrating the principles of the present disclosure. It is to be understood that, in some instances, various aspects of the described implementations may be shown exaggerated or enlarged to facilitate an understanding of the described implementations. In the drawings, like reference characters throughout the various drawings generally refer to functionally similar and/or structurally similar components.

FIG. 1 depicts various stages of a typical clone screening process.

FIG. 2 is a simplified block diagram of an example system that may implement the techniques of a first aspect of the invention described herein.

FIG. 3 is a block diagram illustrating machine learning model inputs and outputs for predicting clone performance.

FIG. 4 is a table illustrating example clone scores scored by a machine learning algorithm in accordance with some implementations.

FIG. 5 is a flow diagram of an example method for predicting clone performance and selecting best-performing clones for production.

FIG. 6A illustrates use of a virtual clone in a machine learning process.

FIG. 6B illustrates a pipeline applying digital twins to develop upstream biologics product development.

FIG. 7A illustrates an example of using digital twins to predict impact of nutrient spikes on a first product quality (PQ1).

FIG. 7B illustrates an example of using digital twins to predict impact of nutrient spikes on a second product quality (PQ2).

FIG. 7C illustrates an example of using digital twins to predict impact of nutrient spikes on a third product quality (PQ3).

FIG. 8 illustrates a machine learning pipeline for predicting top-producing clones in a cell line development process.

FIG. 9 illustrates a system for anomaly detection in fluorescent assay images using an ensemble of machine learning models.

FIG. 10A depicts a graph illustrating a growth curve fit over time using an exponential growth model.

FIG. 10B depicts a comparison of data scaling approaches applied to time series data.

FIG. 11 is a flow diagram of an example method for cell clone assessment using a plurality of machine learning models.

### DETAILED DESCRIPTION

The various concepts introduced above and discussed in greater detail below may be implemented in any of numerous ways, and the described concepts are not limited to any particular manner of implementation. Examples of implementations are provided for illustrative purposes.

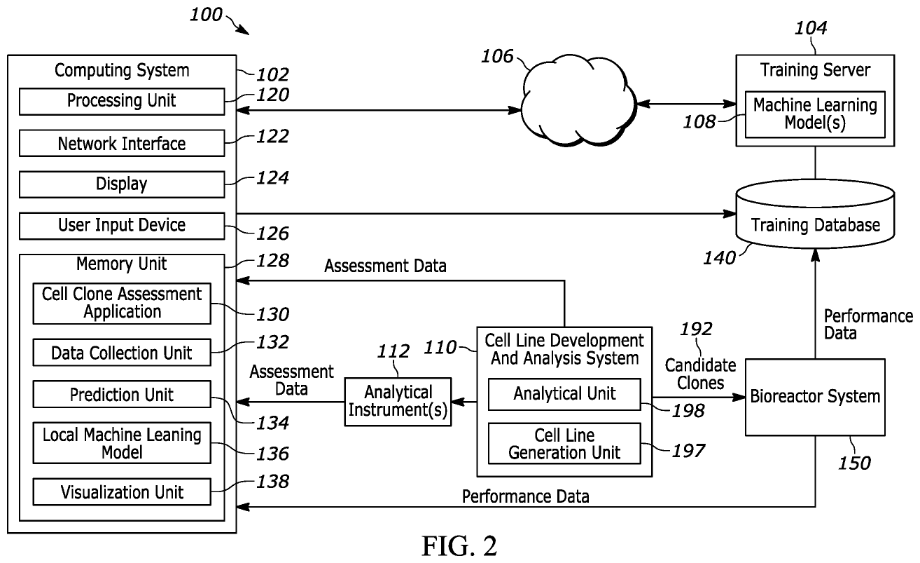

FIG. 2 is a simplified block diagram of an example system 100 that may implement the techniques of the first aspect described herein. System 100 includes a computing system 102 communicatively coupled to a training server 104 via a network 106. Generally, computing system 102 is configured to predict clone performance of cell clones that are output by a cell line development and analysis system 110 using one or more machine learning (ML) models 108 trained by a training server 104.

Network 106 may be a single communication network or may include multiple communication networks of one or more types (e.g., one or more wired and/or wireless local area networks (LANs), and/or one or more wired and/or wireless wide area networks (WANs) such as the Internet). In various embodiments, training server 104 may train and/or utilize ML model(s) 108 as a “cloud” service (e.g., Amazon Web Services), or training server 104 may be a local server. In the depicted embodiment, however, ML model(s) 108 is/are trained by server 104, and then transferred to computing system 102 via network 106 as needed. In other embodiments, one, some or all of ML model(s) 108 may be trained on computing system 102 and then uploaded to server 104. In still other embodiments, computing system 102 trains and maintains/stores the model(s) 108, in which case system 100 may omit both network 106 and training server 104.

FIG. 2 depicts a scenario in which computing system 102 makes predictions of cell clone performance based on cell clones input from a cell clone generation and analysis system 110. Cell clone generation and analysis system 110 may be any suitable (preferably high-throughput) subcloning system. In some embodiments, the clone generation and analysis system 110 is a Berkeley Lights Beacon system. As seen in FIG. 2, the cell clone generation and analysis system 110 includes an analytical unit 198 and a cell line generation and growth unit 197. Cell line generation and growth unit 197 may be a culturing chip containing a plurality of physically isolated pens or wells perfused by microfluidic channels. The unit 197 may be an OptoSelect™ Berkeley Lights chip, for example. Each of the pens may receive a transfected cell from a cell pool with the aid of projected light patterns that activate photoconductors, which repel cells to manipulate those cells (e.g., as provided by Berkeley Lights' OptoElectro™ positioning technology), and contain the cell (and other generated cells of the cell line) throughout a cell line generation and analysis process. Each of cell pool(s) may be a pool of transfected cells (e.g., Chinese hamster ovary (CHO) cells) within a single container, such as a well or vial, for example. The cell pool(s) may be any suitable pool(s) of cells, scaled up through successive cell passages in selective growth media, that produce recombinant proteins, and may be of any modality. The cells may be cells that produce a recombinant protein such as a monoclonal antibody (mAb), or cells that produce a recombinant protein such as a bispecific or other multispecific antibody, for example. Generally, however, the cells of each of pool(s) are not all clonally derived.

Analytical unit 198 of the cell line generation and analysis system 110 is configured to measure physical characteristics of cells in clone generation and growth unit 197. The analytical unit 198 may include one or more sensors or instruments to obtain the measurements directly, and/or may obtain or facilitate indirect or “soft” sensor measurements. Instruments of the analytical unit 198 may include instruments that are fully automated, and/or instruments that require human assistance. As just one example, instruments of the analytical unit 198 (e.g., sensors or other instruments integrated within, or interfacing with, unit 197) may include one or more imaging devices (e.g., a camera and/or microscope) and associated software configured to directly or indirectly measure cell count or cell growth, one or more devices configured to directly or indirectly measure cell productivity by performing secretion assays (e.g., diffusion-based fluorescence assays that bind to antibodies produced by the cells on the chip, such as a secretion assay using a Spotlight Hulg2 Assay (or Spotlight Assay)), and so on.

One or more analytical instruments 112 are configured, collectively, to obtain the physical measurements that will be used by computing system 102 to make predictions, as discussed further below. Analytical instrument(s) 112 may obtain the measurements directly, and/or may obtain or facilitate indirect or “soft” sensor measurements. As used herein, the term “measurement” may refer to a value that is directly measured/sensed by an analytical instrument (e.g., one of instrument(s) 112), a value that an analytical instrument computes based on one or more direct measurements, or a value that another device (e.g., computing system 102) computes based on one or more direct measurements. Analytical instrument(s) 112 may include instruments that are fully automated, and/or instruments that require human assistance. As just one example, analytical instrument(s) 112 may include one or more chromatograph devices (e.g., devices configured to perform size exclusion chromatography (SEC), cation exchange chromatography (CEX), and/or hydrophilic-interaction chromatography (HILIC)), one or more devices configured to obtain measurements for determining titer for a target product, one or more devices configured to directly or indirectly measure metabolite concentrations of the culture medium (e.g., glucose, glutamine, etc.), and so on.

Computing system 102 may be a general-purpose computer that is specifically programmed to perform the operations discussed herein, or computing system 102 may be a special-purpose computing device. As seen in FIG. 2, computing system 102 includes a processing unit 120, a network interface 122, a display 124, a user input device 126, and a memory unit 128. In some embodiments, however, computing system 102 includes two or more computers that are either co-located or remote from each other. In these distributed embodiments, the operations described herein relating to processing unit 120, network interface 122 and/or memory unit 128 may be divided among multiple processing units, network interfaces and/or memory units, respectively.

As seen in FIG. 2, system 100 may further include a bioreactor system 150 communicatively coupled to the computing system 102 and/or the training database 140. The bioreactor system 150 may be a scaled-up bioreactor system, such as an automated micro bioreactor (AMBR) system. The bioreactor system 150 may be configured to culture candidate clones 192 that the computing system 102 selects based on predictions using assessment data from the cell line development and analysis system 110 and/or analytical instruments 112. In some embodiments, the bioreactor system 150 cultures candidate clones 192 in bioreactor vessels of various scales (e.g., 15 mL or 250 mL vessels) over a culture period (e.g., a 13-day or 15-day fed-batch process) and measures performance data including titer values at one or more time points during the culture period (e.g., at Day 13 and/or Day 15 of the culture). The bioreactor system 150 may support replicate runs, wherein a plurality of bioreactor vessels culture the same candidate clone in parallel, thereby providing more robust performance measurements.

In some embodiments, the bioreactor system 150 includes bioreactor vessels at multiple scales. For example, the bioreactor system 150 may include a first set of bioreactor vessels at a smaller scale (e.g., 15 mL) and a second set of bioreactor vessels at a larger scale (e.g., 250 mL). Performance data from the bioreactor system 150 may include titer values, viable cell density values, specific productivity values, and/or purity values measured at each scale. In some embodiments, the bioreactor system 150 measures performance data at multiple time points (e.g., Day 13 and Day 15) and records the number of replicate runs and the standard deviation across replicates for each candidate clone. Prior to culturing in the bioreactor system 150, candidate clones 192 may pass through one or more intermediate stages, such as a selection stage in which candidate clones are selected based on confluence measurements, and/or a fed-batch stage in which candidate clones are cultured in small-scale containers (e.g., spin tubes or deep well plates) and measured for titer, integral of viable cell density (IVCD), and specific productivity (qP).

In some embodiments, the machine learning model 136 is configured to predict performance of candidate clones 192 at the bioreactor system 150 based on assessment data obtained from the cell line development and analysis system 110. That is, the cell line development and analysis system 110 may serve as a cell line screening system at a first stage of a cell line development pipeline, providing early-stage assessment data (e.g., cell morphology, cell growth, secretion assay data, and image data that the system collects during the first several days of cell culture on a microfluidic chip). The bioreactor system 150 may serve as a bioreactor system at a subsequent stage of the cell line development pipeline, providing later-stage performance data (e.g., titer values measured at Day 15 of a scaled-up bioreactor culture) for candidate clones 192 that have been advanced from the cell line development and analysis system 110. The training server 104 may train the machine learning model 136 using the early-stage assessment data from the cell line development and analysis system 110 as input features and the later-stage performance data from the bioreactor system 150 as ground truth data. In this manner, the machine learning model 136 applies experimental knowledge from early-stage screening to predict later-stage bioreactor performance, enabling informed clone selection decisions earlier in the cell line development pipeline.

In some embodiments, the training database 140 includes historical assessment data and corresponding bioreactor performance data from a plurality of historical programs spanning a plurality of molecule modalities (e.g., monoclonal antibodies (mAbs), bispecific antibodies, multispecific antibodies, and other modalities such as immunoglobulin G variable heavy chain (IgG-VH), antibody-antigen binding fragment (AbFrab), antigen binding fragment-fragment crystallizable region (FrabFc), and half-life extended bispecific T-cell engager (HLE BiTE) molecules). For example, the training database 140 may include data from ten or more historical first-in-human (FIH) programs encompassing data from tens of thousands of individual cell clones. Training the machine learning model 136 across multiple programs and modalities may enable the model to generate modality-agnostic predictions that generalize across different molecule types without requiring modality-specific retraining.

In some embodiments, the machine learning model 136 is configured to account for program-to-program variation in assay measurements. For example, different programs may exhibit different levels of impurities and different assay variability due to differences in molecule characteristics, culture conditions, and analytical methods. The machine learning model 136 may normalize or otherwise adjust for such variation during training (e.g., on a per-bioreactor, pre-run, and/or per-modality basis) to generate training data with a common frame of comparison, For example by training on normalized data from multiple programs the model may be able to distinguish biological differences in clone performance across technical or program-specific variations.

In some embodiments, the training server 104 updates the machine learning model 136 over time by supplementing the historical assessment data in the training database 140 with assessment data and corresponding performance data from one or more subsequent programs as those programs complete bioreactor runs at the bioreactor system 150. As seen in FIG. 2, the bioreactor system 150 may store new performance data at the training database 140, enabling continuous improvement of the machine learning models through incorporation of additional performance outcomes from scaled-up processes for clones selected using the techniques disclosed herein. As additional programs complete and the training database 140 incorporates their bioreactor performance data, the predictive accuracy of the machine learning model 136 may improve over time. It should be appreciated that updating the machine learning model 136 may include providing the performance data to the training server 104 for retraining or fine-tuning the machine learning model 136, and that the act of providing such performance data to the training server 104 or the training database 140 may itself constitute updating the machine learning model 136.

In some embodiments, a standardized data pipeline automatically collects and processes data from the cell line development and analysis system 110, the bioreactor system 150, and any intermediate stages. The data pipeline may automate greater than 90% of data collection and processing, including extraction of morphological features from images, extraction of measurements from the cell line development and analysis system 110, and retrieval of bioreactor performance data from the bioreactor system 150. The automated data pipeline may support stage-wise data ingestion, enabling the computing system 102 to generate predictions as assessment data becomes available at each stage of the cell line development pipeline.

By training the machine learning model 136 using early-stage assessment data from the cell line development and analysis system 110 as input features and later-stage performance data from the bioreactor system 150 as ground truth data, the system 100 may learn the correlations between early-stage features and later-stage performance, thereby providing improvements over conventional clone screening processes. Conventional approaches may require advancing hundreds or thousands of candidate clones through resource-intensive bioreactor runs at the bioreactor system 150 to identify top-performing clones, a process that may take several months and consume significant laboratory resources. In contrast, the system 100 may predict bioreactor performance based on early-stage screening data that the cell line development and analysis system 110 collects within the first several days of cell culture, thereby reducing the number of candidate clones that need to advance to the bioreactor system 150 and enabling clone selection earlier in the development pipeline. In some embodiments, the system 100 may reduce the number of clones requiring bioreactor screening by 60% or more, while maintaining or exceeding the accuracy of manual subject matter expert selection. For example, the machine learning model 136 may achieve a performance metric (e.g., an enrichment score an accuracy score, or other suitable performance metric) of 0.722 in identifying top-performing clones at the colony growth stage, compared to a performance score of 0.612 for manual expert selection. In some embodiments, model performance may be validated using leave-one-out cross-validation across diverse programs, wherein each program is held out in turn and the model is trained on the remaining programs, enabling assessment of generalization performance across different molecule types and experimental conditions.

In some embodiments, the computing system 102 is configured to automatically select, based on the clone performance predictions, a subset of candidate clones 192 for advancement to the bioreactor system 150. For example, the computing system 102 may automatically apply an advancement label to data objects associated with the top-ranked candidate clones, configure the bioreactor system 150 to receive and culture the selected candidate clones 192, and/or generate instructions for an automated liquid handling system to transfer the selected candidate clones 192 from the cell line development and analysis system 110 to the bioreactor system 150. In some embodiments, the system 100 provides early predictions of top-producing clones at one or more stages of the cell line development process, including at a single-cell cloning stage (e.g., Day 3 or Day 4 of single-cell cloning), a colony growth stage, and/or a cherrypick stage. These early predictions may guide experimental teams in prioritizing the most promising candidates for validation, reducing timelines and improving decision-making in process development.

In some embodiments, the computing system 102 is communicatively coupled to the bioreactor system 150 and is configured to control one or more operations of the bioreactor system 150. For example, the computing system 102 may output instructions for controlling the bioreactor system 150 when performing a culture run for one or more selected candidate clones 192. For example, the instructions may indicate an adjustment to culture parameters (e.g., temperature, pH, dissolved oxygen, feed rates) during the culture run, and/or define a timeframe for performing culture run. In some embodiments, the computing system 102 and the bioreactor system 150 are controlled by a common entity, and the computing system 102 automatically configures the bioreactor system 150. In other embodiments, the computing system 102 may output instructions for a user to manually configure the bioreactor system.

The computing system 102 may also provide one or more outputs to facilitate advancement of selected cells from the cell line development and analysis system 110 to the bioreactor system 150. For example, the computing system may output a list of well or pen identifiers housing candidate clones to be advanced. As another example, the computing system may output a map or grid that visually represents the physical location of the selected candidate clones with respect to a well plate in which the candidate clones are developed within the cell line development and analysis system 110. As a result, an operator is more readily able to identify a location of the candidate clones that were selected for advancement.

The bioreactor system 150 may perform one or more culture operations on the selected candidate clones 192, including inoculating bioreactor vessels with the selected candidate clones 192, culturing the selected candidate clones 192 under controlled conditions, sampling the culture medium at predetermined intervals, and measuring performance data including titer, viable cell density, and product quality attributes. The performance data generated by the bioreactor system 150 may be automatically transmitted back to the computing system 102 and/or the training database 140 for use in updating the machine learning models.

In some embodiments, the machine learning model(s) 108 include(s) a plurality of stage-specific models, each stage-specific model trained using assessment data captured at a different timepoint during a cell line screening process performed by the cell line development and analysis system 110. For example, a first stage-specific model may accept as input assessment data captured at a Day 3 single-cell cloning timepoint, a second stage-specific model may accept as input assessment data captured at a Day 4 single-cell cloning timepoint, and a third stage-specific model may accept as input assessment data captured at a colony growth timepoint. Because additional assessment data becomes available as the cell line screening process progresses, a stage-specific model trained using assessment data captured at a later timepoint may receive a larger set of features than a stage-specific model trained using assessment data captured at an earlier timepoint. For example, a model trained on Day 4 assessment data may incorporate time-dependent morphological features, growth kinetics features, and secretion assay data that were not yet available at Day 3. As such, the models that accept input features sets including assessment data later in the cell line development process may be more accurate. On the other hand, collecting this later assessment data involves waiting until the later time, introducing delay in the clone selection process. The computing system 102 may select the appropriate stage-specific model(s) based on user-or system-provided timing data, thereby generating predictions tailored to balance the tradeoff between accuracy and time. Additionally, different model types (or ensembles thereof) may have different predictive performance based on the timing of the assessment data. Thus, the computing system 102 may ensure alignment between the indicated timing data and the models used to perform the predictions. Hardware and software components of computing system 102, including processing unit 120 and memory unit 128 described in more detail below, may implement the foregoing machine learning techniques.

Processing unit 120 includes one or more processors, each of which may be a programmable microprocessor that executes software instructions stored in memory unit 128 to execute some or all of the functions of computing system 102 as described herein. Processing unit 120 may include one or more central processing units (CPUs) and/or one or more graphics processing units (GPUs), for example. Alternatively, or in addition, some of the processors in processing unit 120 may be other types of processors (e.g., application-specific integrated circuits (ASICs), field-programmable gate arrays (FPGAs), etc.), and some of the functionality of computing system 102 as described herein may instead be implemented in hardware. Network interface 122 may include any suitable hardware (e.g., a front-end transmitter and receiver hardware), firmware, and/or software configured to communicate with training server 104 via network 106 using one or more communication protocols. For example, network interface 122 may be or include an Ethernet interface, enabling computing system 102 to communicate with training server 104 over the Internet or an intranet, etc.

In some embodiments, the machine learning model(s) 108 include multiple modeling strategies that are applied and combined through ensemble learning. For example, the machine learning model(s) 108 may include one or more classification models, one or more regression models, and one or more pairwise comparison models. A classification model may be configured to classify candidate clones with respect to a clone performance metric (e.g., classifying a clone as being predicted to meet or not meet a performance threshold). A regression model may be configured to predict an absolute value for a clone performance metric (e.g., predicting a titer value at a predetermined culture day, such as Day 15 titer). A pairwise comparison model may be configured to compare pairs of candidate clones and predict which clone of the pair has a superior clone performance metric, as described in further detail below. In some embodiments, two or more of these modeling strategies are applied in combination. That is, in some embodiments, ensemble models may be generated of the same model type (e.g., multiple regression models trained to accept assessment data having different timing and/or to predict different values), across model types (e.g., an ensemble of classification and regression models), and/or a combination thereof (e.g., an ensemble of ensemble models). In some embodiments, a classification model may use a hybrid approach that combines classification with additional post-processing techniques to account for pool diversity. In some embodiments, the machine learning model(s) 108 may include a gradient boosting ranker model (e.g., an XGBRanker) configured to rank candidate clones based on learned feature importance.

In some embodiments, the classification model comprises a multi-layer perceptron (MLP) neural network classifier. In some embodiments, the classification model comprises a support vector machine (SVM) classifier. In some embodiments, the regression model comprises a multi-layer perceptron (MLP) neural network regressor. In some embodiments, the pairwise comparison model comprises a Siamese neural network configured to process features of both candidate clones of a pair through shared network weights, thereby reducing computational overhead by handling all pairwise comparisons with a single network architecture. The Siamese neural network may identify meaningful differences between inputs, making the model more robust to noise in the data. In other embodiments, the pairwise comparison model may calculate feature differences between paired clones or concatenate features of paired clones as input to a classification model.

In some embodiments, the machine learning model(s) 108 include(s) ensemble variants of one or more of the classification models, regression models, and/or pairwise comparison models. An ensemble variant may combine predictions from multiple base model instances of the same model type (e.g., multiple MLP classifier instances) to provide improved robustness and stability over single model predictions. The ensemble approach may use the diversity of multiple trained models to reduce prediction variance and improve overall prediction performance. In some embodiments, the ensemble variants may be developed to enhance prediction performance and stability through model combination techniques.

In some embodiments, the machine learning model(s) 108 include a decision fusion layer configured to generate ensemble predictions by combining results from different base model types. For example, the decision fusion layer may combine predictions from a classification model and a regression model to generate ensemble predictions that combine the strengths of different modeling strategies. The decision fusion layer may take individual model results and specified model pair combinations as input, create ensemble predictions by pairing different model types (e.g., classification and regression), and merge the combined results with original results. In some embodiments, combining outputs from different model types comprises selecting candidate clones for the output based on respective outputs from the classification model and the regression model. The ensemble predictions may enable evaluation of whether hybrid approaches outperform individual models for identifying top-performing clones. The decision fusion layer may also be referred to as a fusion model. In some embodiments, the fusion model selects highest performing candidate clones with respect to each of the two or more machine learning models. The outputs of the fusion model, including clone rankings and selections, may be presented to a user via display 124 and other output components of computing system 102.

Display 124 may use any suitable display technology (e.g., LED, OLED, LCD, etc.) to present information to a user, and user input device 126 may be a keyboard or other suitable input device. In some embodiments, display 124 and user input device 126 are integrated within a single device (e.g., a touchscreen display). Generally, display 124 and user input device 126 may combine to enable a user to interact with graphical user interfaces (GUIs) provided by computing system 102. For example, the display 124 can enable a user to view outputs indicate of best-performing candidate clones as described later herein. In some embodiments, however, computing system 102 does not include display 124 and/or user input device 126, or one or both of display 124 and user input device 126 is/are included in another computer or system (e.g., a client device) that is communicatively coupled to computing system 102.

Memory unit 128 may include one or more volatile and/or non-volatile memories. Any suitable memory type or types may be included, such as read-only memory (ROM), random access memory (RAM), flash memory, a solid-state drive (SSD), a hard disk drive (HDD), and so on. Collectively, memory unit 128 may store (e.g., on a non-transitory computer-readable medium) one or more software applications, the data received/used by those applications, and the data output/generated by those applications. These applications include a cell clone assessment application 130 that, when executed by processing unit 120, predicts cell clone performance by applying assessment data as inputs to a machine learning model configured to predict a clone performance metric based on measurements obtained by analytical instrument(s) 112 (and possibly also based on other information, such as modality). While various modules of application 130 are discussed below, it is understood that those modules may be distributed among different software applications, and/or that the functionality of any one such module may be divided among two or more software applications.

A data collection unit 132 of application 130 collects values of various assessment data. For example, the data collection unit 132 may receive measurements or assessment data directly from the cell line development and analysis system 110 or from the cell line development and analysis system 110 via the analytical instrument(s) 112. Additionally or alternatively, the data collection unit 132 may receive information stored in a measurement database (not shown in FIG. 2) and/or information entered by a user (e.g., via user input device 126). For example, the data collection unit 132 may receive a modality, target drug product, drug protein scaffold type, and/or any other suitable information entered by a user and/or stored in a database. Additionally or alternatively, the data collection unit 132 can obtain cell growth, image data, phenotypical and secretion data of historical and future programs of the cell line development and analysis system 110. Additionally or alternatively, data collection unit may receive measurements from an opto-electronic device as described herein.

A prediction unit 134 of application 130 operates on the values collected by the data collection unit 132 to output one or more predictions related to clone performance metrics, for candidate clones and pairs of candidate clones (as described later herein). using a local machine learning model 136. That is, the assessment data collected by the data collection unit 132 are used as inputs/features for the machine learning model 136. The assessment data can include attribute(s) for which value(s) is/are predicted may include one or more productivity metrics (e.g., titer) and/or one or more product quality metrics (e.g., SEC main peak, low molecular weight peak, and/or high molecular weight peak percentage(s). The machine learning model 136 may be a classification model configured to receive, as an input, a set of features associated with two different pens, and output a prediction of which pen is predicted to exhibit superior performance with respect to the productivity metrics and/or the product quality metrics. In some embodiments, the machine learning model 136 associates the superior pen with a 1 and the inferior pen with a 0. For some input features, the values may be obtained from the analytical instruments 112 and/or the cell line development and analysis system 110. For other input features, the values may be derived by applying a feature extraction model to image data generated by the analytical instruments 112 and/or the cell line development and analysis system 110. It should be appreciated that when referencing a set of data associated with a particular well or pen that includes a cell colony derived from a clone, the terms “clone”, “cell colony,” and “well” or “pen” may be used interchangeably.

In the depicted embodiment, machine learning model 136 includes a local copy of one of the model(s) 108 trained by training server 104, and may be stored in a RAM of memory unit 128, for example. The models 108 may include the classification models and/or any feature extraction models. As noted above, however, the server 104 may utilize all models 108 in other embodiments, in which case no local copy need be present in the memory unit 128.

It should be appreciated that in instant approaches, the machine learning model 136 does not generate a prediction of an absolute value for the productivity metrics and/or the product quality metrics. Instead, the machine learning model 136 may be configured to determine a relative value for the productivity metrics and/or the product quality metrics. That is, rather than outputting a predicted titer value, the machine learning model 136 may be configured to accept as an input feature sets associated with two different pens and output an indication of which pen is predicted to have a higher value for the productivity metrics and/or the product quality metrics.

This relative approach has several benefits. A first benefit is that accurately predicting a value for the productivity metrics and/or the product quality metrics can be difficult and require large training sets to account for the various factors that influence the productivity metrics and/or the product quality metrics. Often, sufficient data is unavailable, resulting in lower accuracy in the predicted value. Given the inaccuracies in these predictions, personnel analyzing the predictions to advance the most promising cell colonies tend to look at the predicted values as a benchmark for predicted relative performance, as opposed to gaining an understanding of the predicted absolute value of the productivity metrics and/or the product quality metrics of interest.

While these training sets may be insufficient to predict absolute values with a desired accuracy, the training sets may still be large enough to predict which pen is predicted to have superior performance with respect to the productivity metrics and/or the product quality metrics from among a pair of pens. That is, if a well plate or similar holder has n pens with viable cell colonies, performing n! comparisons and counting the number of times each pen was predicted to have the superior performance results in the ability to sort the pens based on relative predicted performance without the need to accurately predict a particular value for the productivity metrics and/or the product quality metrics. Thus, the prediction model can be trained using smaller sets of training data while still providing an accurate metric by which cells can be advanced to a subsequent stage. The ability to rely on smaller set of training data may also enable the ability to train a model specific to different parent pool of clones, resulting in increased predictive accuracy.

Operation of system 100, according to one embodiment, will now be described in further detail, for the specific scenario in which application 130 is used to predict cell clone performance based on data provided by cell line development and analysis system 110. By so doing, a reduced number of clones corresponding to those with the highest predicted relative performance can be selected to enter next stages of screening.

Initially, training server 104 trains machine learning model(s) 108 using data stored in a training database 140. Machine learning model(s) 108 may include a number of different types of machine learning based regression estimators (e.g., a decision tree regressor model, a random forest regressor model, a linear support vector regression model, an extreme gradient boosting (xgboost) regressor model, etc.), and possibly also one or more models not based on regression (e.g., a neural network). Moreover, model(s) 108 may include more than one model of any given type (e.g., two or more models of the same type that are trained on different historical datasets and/or using different feature sets), in some embodiments. Each of machine learning models 108 may be optimized (trained and tuned) using, for example, cross-validation, re-training, and hyperparameter tuning. Moreover, each of machine learning models 108 may rank clones within parental pools based on correlations between independent features and the fed-batch titer. That is, the model data for the classification model may be tailored to each parental pool. Machine learning model(s) 108 can include image analysis models configured to output cell characteristics including cell aggregation, cell morphology, cell size, and cell shape.

Training database 140 may include a single database stored in a single memory (e.g., HDD, SSD, etc.), or may include multiple databases stored in one or more memories. For each different model within machine learning model(s) 108, training database 140 may store a corresponding set of training data (e.g., input/feature data, and corresponding labels), with possible overlap between the training data sets. To train a model that predicts relative clone performance, for instance, training database 140 may include data provided by the cell line development and analysis system 110 or measurements made by analytical instruments (e.g., analytical instrument(s) 112 and/or similar instruments), and possibly other information (e.g., modality), along with a label for each cell colony. In some embodiments, the training data is based on historical cell line development processes. For example, the historical data sets may includes both the analytical data used to derive the input feature sets and the ultimate values for the productivity metrics and/or the product quality metrics at the end of the subsequent stage of the cell line development process. Accordingly, the training data may be generated by obtaining and/or performing the calculations to generate the corresponding feature sets for each pen in the historical data. The training process may then select pairs of historical pen data and use the ultimate values for the productivity metrics and/or the product quality metrics as the ground truth data for identifying the superior performing pen. It should be appreciated that if the historical data for a particular pen is missing data from the data set, that pen may be excluded from the training data.

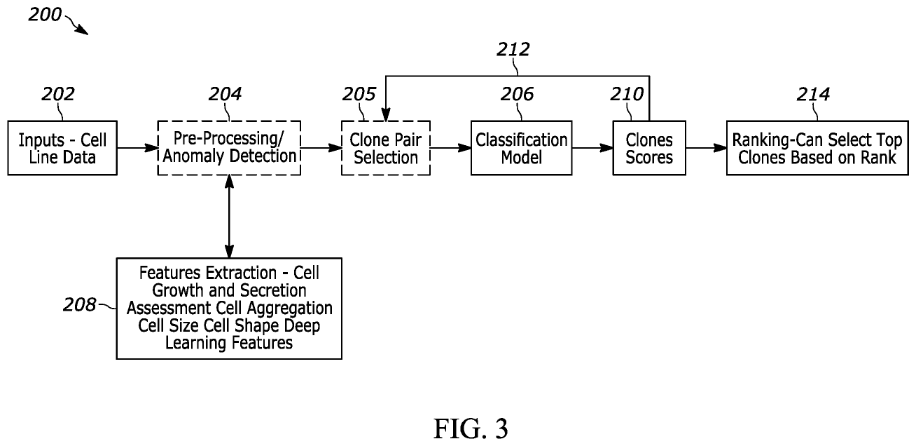

Other models can extract quantitative phenotype data (or other feature values) from single-cell image data as described in more detail with reference to FIG. 3 to derive features values included in the input feature set for the classification model. Application 130 may retrieve from training server 104 via network 106 and network interface 122, a specific one of machine learning models 108. Upon retrieving the model, computing system 102 stores a local copy as local machine learning model 136. In other embodiments, as noted above, no model is retrieved, and input/feature data is instead sent to training server 104 (or another server) as needed to use the appropriate model of model(s) 108.

In accordance with the feature set used by the model 136, the data collection unit 132 collects the necessary data. For example, the data collection unit 132 may communicate with analytical instrument(s) 112 to collect measurements of titer, chromatography values, metabolite concentrations, and/or other specific attributes of different clones retrieved via the cell line development and analysis system 110. As another example, the data collection unit 132 may collect data directly from the cell line development and analysis system 110. In one such embodiment, the data collection unit 132 sends commands to one or more of analytical instrument(s) 112 to cause the one or more instruments to automatically collect the desired measurements.

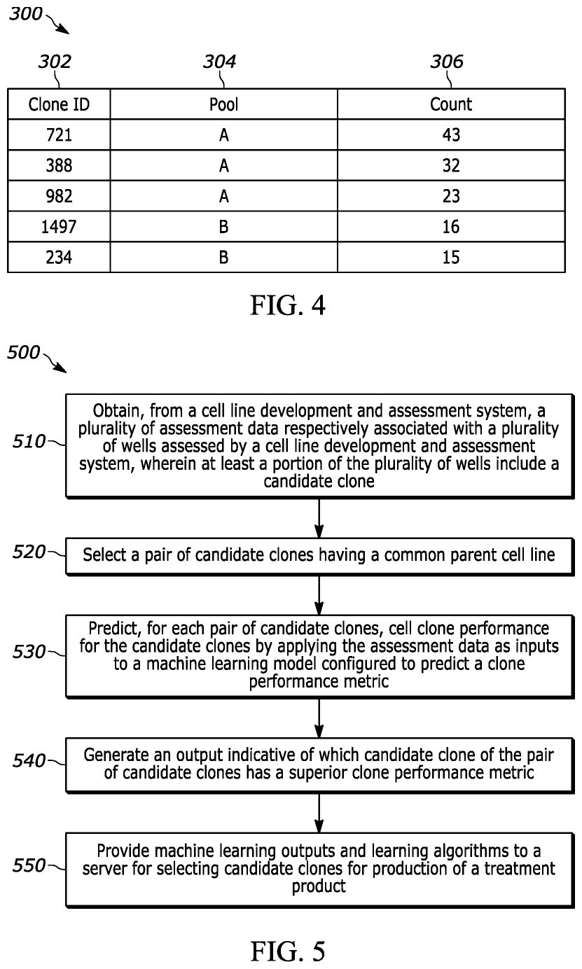

After the data collection unit 132 has collected the assessment data, the data collection unit 132 inputs assessment data to the prediction unit 134 which causes model 136 to operate on those inputs to output a prediction of relative clone performance with respect to the productivity metrics and/or the product quality metrics. Clones can then be ranked, as described in more detail later herein with reference to FIG. 4.

Other machine learning models can determine other attributes of clones, clone pairs, and clone pools and store the attributes for later analysis and learning. For example, attributes can include one or more productivity attributes such as titer or viable cell density (VCD), and/or one or more product quality attributes such as SEC main peak (MP) percentage, SEC low molecular weight (LMW) peak percentage, and/or SEC high molecular weight (HMW) peak percentage. These attributes can be used in later programs that may make use of clones, clone pairs or parental pools as generated or provided by the cell line development and analysis system 110. Visualization unit 138 causes a user interface, depicted on display 124, to present information regarding superior/inferior clones, parental pool-wide attributes, and/or other information derived from the predicted clone performance metrics or related data and attributes. For example, visualization unit 138 may cause the user interface to present an indication of clone candidate rankings.

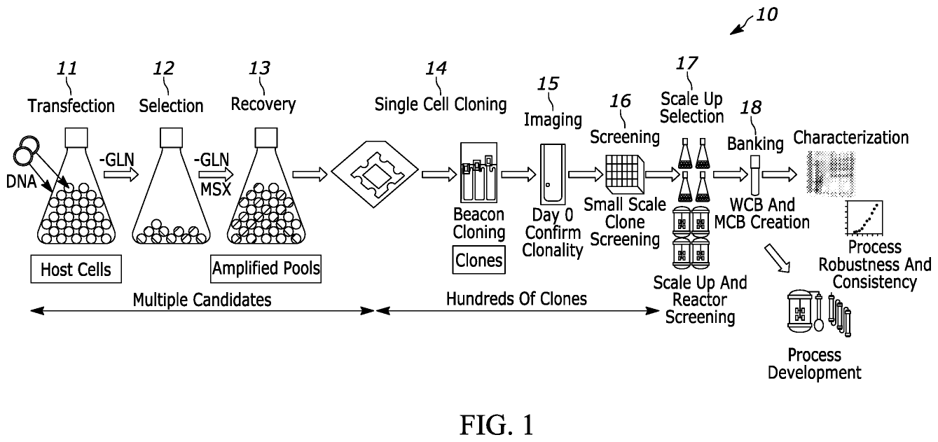

Prediction unit 134 may store the predictions made by model 136 for each clone, each clone within a particular parental pool, or other grouping in memory unit 128 or another suitable memory/location. The prediction unit 134 can also store learned cell characteristics such as cell aggregation, morphology, cell growth features, cell size, cell shape, and image classification features. After predictions have been made and stored, a subset of the clones may be selected for scale-up (e.g., similar to stage 17 of FIG. 1). Again, this selection may be fully automated according to some criteria specific to the use case, or selection may involve human interaction. A “use case” may be defined in various ways, in a manner that determines which data is relevant to that use case. For example, a use case may be designed to understand the impact of increasing trace metal concentration in a batch medium and increasing cell-specific production on various PQ attributes for a commercial product. Such PQ attributes can include antibody stability, efficacy, safety, and potency. Machine learning models therefore can be trained to predict relative PQ for completed and predicted lots.

Various techniques for selecting candidate clones and for identifying the most predictive features for a given model or use case, are now described with reference to FIGS. 3 through 5.

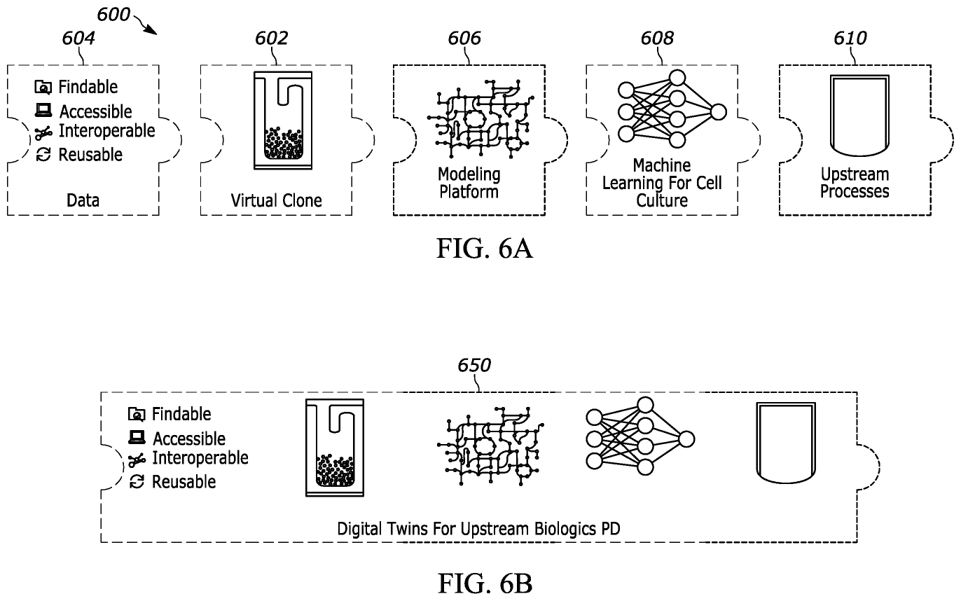

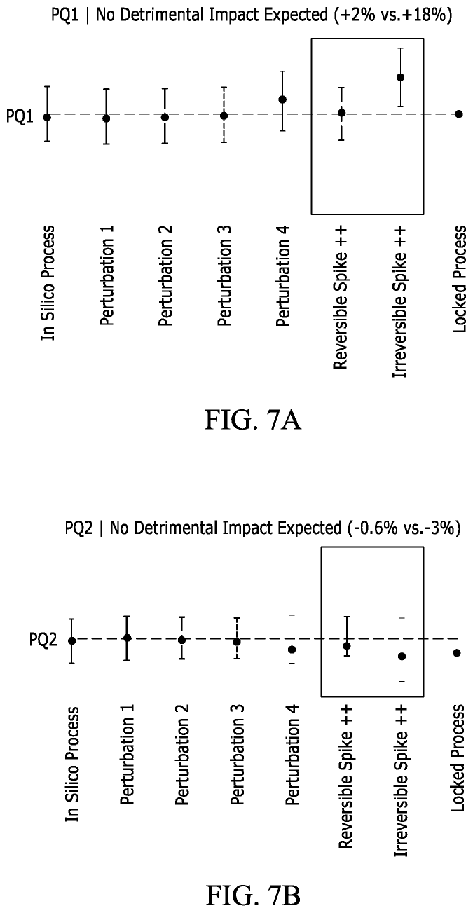

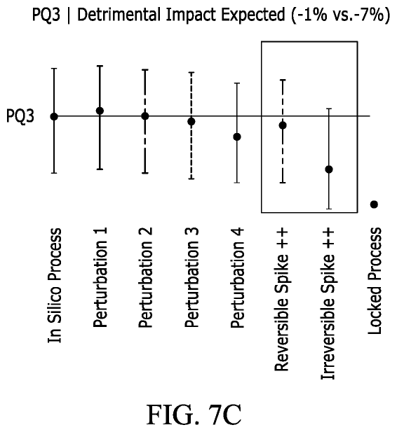

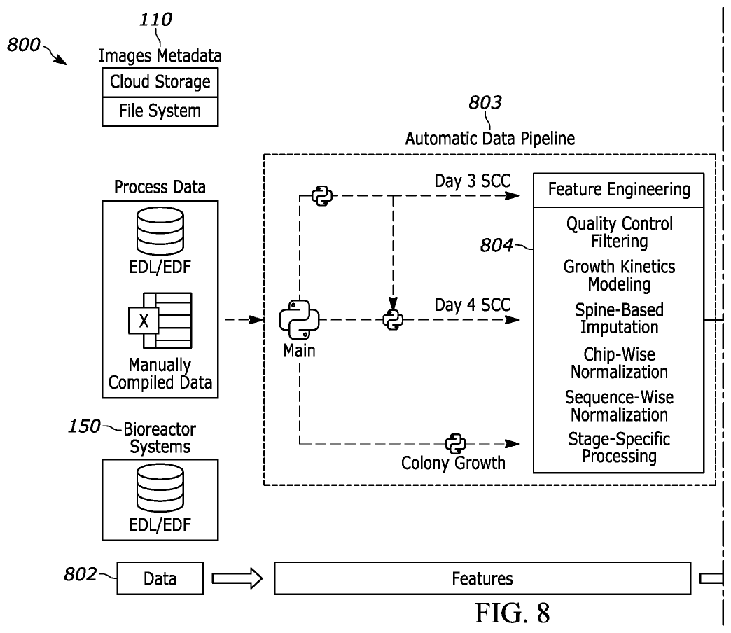

Generally, well-performing models for specific use cases may be identified by training a number of different models using historical training data generated from previous clone screening runs and comparing the results. The historical data may include data of transfected cells (e.g., Chinese hamster ovary (CHO) cells) collected from previous programs. In some embodiments, the historical data is supplemented with digital “twins” (e.g., artificially generated well data designed to mimic desired characteristics of a real pen that exhibits those characteristics). These digital twins can be generated based on knowledge from historical molecules that exhibit the desired characteristics to inform later development programs as described later herein with reference to FIGS. 6A-7C. In some embodiments, the image data associated with a digital twin is generated using feature transposition techniques and/or a diffusion or other image generation model.

Regardless, the historical data may include both categorical data, such as media type and modality, and numerical data, such as metabolite concentrations and titer values. Data for clones that do not originate from a single cell can be filtered out. For small-scale cell line development data (also referred to herein as simply “cell line development data” or “CLD data” such as growth and secretion data), growth factors such as viability, VCD and glucose concentrations may be collected periodically over time (e.g., at different days of a 10-day culture). For scaled-up bioreactor data (also referred to herein as “bioprocess development data” or “BD data”), these attributes, and possibly additional attributes such as pH level and dissolved oxygen concentration, may be collected and recorded in connection with each feature set. The bioreactor data may also include data that serves as the labels for the various feature sets, such as product titers and other analytic results from assays (e.g., results of SEC and/or CEX analysis). Various measures may be taken to ensure a robust set of training data (e.g., providing standardized, heterogeneous data, removing outliers, imputing missing values, and so on).

In some embodiments, special feature engineering techniques are used to extract or derive useful features. For example, a convolutional neural network (CNN, or an API that automatically extracts summary statistics from temporal data, such as tsfresh) may be used to detect temporal dependencies among various attributes (e.g., a high correlation between VCD at Day 0 of the small-scale culture and VCD at Day 6 of the small-scale culture, etc.). These temporal dependencies may be used to extract/derive useful features for model training. CNNs, support vector machines (SVMs) and autoencoders can be used for anomaly detection in fluorescent images to ensure quality candidates (e.g., in a pre-processing stage to filter out inferior candidate clones). Other feature engineering techniques may also be used, such as analysis of variance (ANOVA), and eliminating features with high covariance, for example.

A process 200 that can be used as a framework for identifying top candidate clones for scale-up is shown in FIG. 3. The process 200 can be performed by components of the computing system 102 or other components shown in FIG. 2. Initially, at stage 202, the computing system 102 can receive cell line data. In some examples, the cell line data is received from the cell line development and analysis system 110 (FIG. 2). As mentioned earlier herein, the cell line development and analysis system 110 may be any suitable (preferably high-throughput) subcloning system.

At stage 204, the computing system 102 can perform pre-processing (including for example anomaly detection or normalization) on the input data. Pre-processing algorithms can reduce or eliminate the presence of poor quality or anomalous clones from consideration in subsequent stages of the process 200. Anomaly detection algorithms can include machine-learning based anomaly detection including CNNs, support vector machines and autoencoders that receive fluorescent assay images as input and that provide outputs indicating presence of anomalies.

Furthermore, normalization can remove non-biological biases from assay results and enable chip-chip comparisons by normalizing scores such that scores have comparable ranges across different cell line generation and analysis system 110 chips. For example, empty pens or wells may exist in some outputs as the result of an imperfect cell loading technique. Empty wells, however, can be used to normalize image data using a rolling median method that calculates a rolling average across multiple wells. Subsequently to this normalization process, empty wells can be removed from further consideration.

At stage 208, the computing system 102 performs feature extraction with respect to the non-empty wells. As described above, the feature extraction process may involve inputting the image data associated with a well into one or more feature extraction models. These feature extraction models may be configured to analyze different characteristics of the cell colony within the well. For example, the feature extraction models may include one or more models configured to identify cell growth and secretion, cell aggregation, cell size, cell shape characteristics, and/or features derived from a deep learning model that may not have a human-interpretable representation. To save processing resources, the computer system 102 may be configured to generate certain features first to act as a filter for pens that are unlikely to exhibit superior performance before calculating other features that are more processor intensive to derive. For example, cell colonies with low aggregation may be unlikely to exhibit superior performance. Accordingly, the computer system 102 may be configured to exclude lowest ranking portion of wells with respect to cell aggregation before generating processor-intensive deep learning features and/or feature derived from fluoroscopic imaging. This exclusion has the additional benefit of reducing the number of clone pairs that need to be assessed. After the computer system 102 may then associate the feature values generated by the feature extraction process 208 with the analytical data associated with the well that was obtained via the analytical instruments 112 and/or the cell line development and analysis system 110.

At stage 205, the computer system 102 selects a pair of wells to compare using the classification model. It should be appreciated that each well that was not excluded via the pre-processing stage 204 and/or the feature extraction stage 208 may need to be compared to each other well. Accordingly, at stage 205, the computer system 102 may track which clone pairs have previously been selected such that only new comparisons are performed. In some embodiments, the computer system 102 may be configured to execute multiple instances of the classification model in parallel. In these embodiments, the computer system 102 may select as many clone pairs as there are instances of the classification model.

At stage 206, the feature sets associated with the selected clone pairs are applied as inputs to the classification model to predict which clone will exhibit superior performance with respect to the productivity metrics and/or the product quality metrics. As described above, the classification model may be a binary classification model that is configured to output a label associated with superior performance or a label associated with inferior performance for both of the input clones. For the sake of aggregation, the label associated with superior performance may be a 1 or converted to a 1, and the label associated with inferior performance may be a 0 or converted to a 0. As described above, the particular classification model to which the inputs are applied may vary depending on the particular parental pool from which the clone derives.

At stage 210, the classification model outputs candidate clone classifications for the clones. As mentioned earlier herein, clones are associated with pens or wells, and each time a clone is classified as the superior clone of a pair, a count for the associated well or pen is incremented. That is, each pen is associated with a count that tracks the number of comparisons for which the clone was predicted to have superior performance with respect to the performance metrics and/or product quality metrics. Accordingly, after the completion of the iterative process 212 to compare all of the pens against each other, the count reflects a metric indicative of predicted relative performance from amongst the pens assessed via the analytical instrument 112 and/or the cell line development and assessment system 110.

At stage 214, the computer system 102 sorts the pens based on their count values to provide a ranked list of pens. The ranked list may then be used to select a predetermined number of cell colonies (e.g., 16, 24, 32, 40, etc.) to advance to a subsequent stage of the cell line development process. For example, the computer system 102 may automatically apply an advancement label to a data object associated with the pen, configure a display to provide an indication of the selected pens, and/or present the list for manual selection of the pens.

An example ranking table 300 is shown in FIG. 4. In the example, the table 300 includes a clone identifier column 302 that identifies a particular clone or pen a parental pool column 304 that identifies the parental pool associated with the clone or pen, and a count column 306 that indicates the output count from stage 214. In the illustrated example, the table 300 is sorted based on the count column 306. Accordingly, clone 721 is predicted to have the best relative performance with respect to the other assessed clones.

In some embodiments, the cell clone assessment application 130 can export an association between candidate clones and clone performance metrics to the training server 104 (or other server) to facilitate training or fine tuning of the classification model based on the outputs of the performance metrics and/or product quality metrics observed in the subsequent stages. If there is any change in relative performance during actual observation, this may be noted as ground truth data used to tune and/or train the classification model. As a result, the techniques disclosed herein relate to a continuous learning process that has improved classification accuracy as additional tests are performed.

As noted above, it may be advantageous to reduce the number of features needed for clone assessment. Therefore, only those features that are most predictive of the desired output (e.g., titer, etc.) may be utilized. To identify the smaller feature set, the process 200 may implement recursive feature elimination (RFE), which allows for recursive reduction of explanatory features that are to be used in the final production model, discarding the least important features. The RFE algorithm trains on the data by utilizing a subset of features to yield optimal model performance with respect to a constraint on the number of features. Pairing RFE with sparsity-inducing models/estimators such as decision trees or elastic net can further reduce the number of explanatory features, in a trade-off that increases interpretability at the expense of model accuracy. Through RFE, an elbow plot can be used to determine the “sweet spot”, or inflection point between interpretability and accuracy.

In some instances, users may wish to test two or more use cases in order to select a winning clone, or to select a set of clones to be scaled-up in bioreactors for further screening. In these instances, application 130 (or a remote server such as server 104) may select and run multiple models that are all used to make large-scale predictions for each clone/cell line. For example, a user may wish to consider both titer and SEC main peak at large-scale when selecting a winning clone. Thus, application 130 may select and/or run a first machine learning model for a use case corresponding to end-point titer (e.g., a random forest model), and a second machine learning model for a use case corresponding to SEC main peak (e.g., an xgboost model). As another example, a user may wish to consider titer, SEC main peak, SEC low molecular weight, and SEC high molecular weight at large-scale when selecting a winning clone, and application 130 may select and/or run a random forest model for titer, an xgboost model for SEC main peak, and a decision tree model for both SEC low molecular weight and SEC high molecular weight.

FIG. 5 is a flow diagram of an example method 500 for clone assessment. The method 500 may be implemented by processing unit 120 of computing system 102 when executing the software instructions of application 130 stored in memory unit 128, or by one or more processors of server 104 (e.g., in a cloud service implementation), for example.

At block 510, the processing unit 120 (comprised of, for example, one or more processors as described earlier herein) obtains, from a cell line development and assessment system, a plurality of assessment data respectively associated with a plurality of wells assessed by a cell line development and assessment system. At least a portion of the plurality of wells include a candidate clone. Assessment data can include image data of the plurality of wells. The processing unit 120 can identify empty wells of the plurality of wells. The processing unit 120 can perform normalization as described earlier herein to normalize image data of wells with candidate cells using image data associated with empty wells. The image data can be visible image data captured via visible light illumination, or fluorescent image data captured via fluorescent imaging, although aspects of this disclosure are not limited thereto. In some examples, the machine learning model comprises a first image analysis model is configured to analyze the visible image data and a second image analysis model configured to analyze the fluorescent image data.

At block 520, the processing unit 120 selects a pair of candidate clones having a common parent cell line. A machine learning model can be selected based on the common parent cell line, wherein the machine learning model used can be any model type described earlier herein.

At block 530, the processing unit 120 predicts, for each pair of candidate clones, cell clone performance for the candidate clones by applying the plurality of assessment data as inputs to a machine learning model (e.g., the machine learning model applied at stage 206 (FIG. 3)) configured to predict a clone performance metric. Winning candidate clones of each pair can be selected for an iterative process for comparison with other winning candidate clones, and the processing unit 120 can continue this iterative process until a set of high-ranking candidate clones is selected, based on one or more desired performance metrics.

The machine learning model can comprise one or more image analysis models configured to output one or more cell characteristics. The cell characteristics can include indications of cell aggregation, cell morphology, cell growth, cell size, cell shape, etc. The machine learning model can combine one or more cell characteristics to generate the clone performance metric.

At block 540, the processing unit generates an output indicative of which candidate clone of the pair of candidate clones has a superior clone performance metric. In example aspects, the clone performance metric is indicative of titer, growth, productivity, and/or cell quality.

The method 500 can conclude with operation 550, wherein the processing unit 120 provides machine learning outputs and learning algorithms to a server (e.g., a production server) for selecting candidate clones of r production of a treatment product. As such, the method 500 can produce AI/ML models to accelerate process understanding and provide improved and faster prediction and control of cell clone quality attributes. Historical molecules can be studied and used in reinforcement learning to inform future development across products and at increased scales. In some aspects, machine learning models described herein can help determine whether and how much nutrient concentration can be reduced without negatively impacting product quality, and whether and how much nutrient spikes impact cell performance.

In some example embodiments, some attributes are measured for a plurality of candidate cell lines using the cell line development and analysis system 110 (FIG. 2). For example, the cell line development and analysis system 110 can perform optical and assay measurements for the candidate cell clones or cell lines. In some embodiments, such measurements are performed, at least in part, by measuring at least cell counts and cell productivity scores at a plurality of physically isolated pens in the cell line development and analysis system 110. In some of these embodiments, methods can include generating cells of the candidate cell lines using the opto-electronic cell line generation and analysis system, at least by moving individual cells into different ones of the physically isolated pens with one or more photoconductors activated by light patterns, and by containing the individual cells within their respective pens throughout a cell line generation and analysis process. Further still, methods can include measuring different values of assessment data or any other measurement data on different days of the cell line generation and analysis process. More generally, the first plurality of attribute values may include values of any of the attributes that can be measured by analytical unit 198 (FIG. 2) as discussed elsewhere herein, and/or may include values of any suitable attributes that can be measured using an opto-electronic cell line generation and analysis system.

In some implementations, methods can include the one or more processors generating a digital representation of at least one virtual clone having a set of predetermined characteristics. The digital representation can include image data. The one or more processors can generate the digital representation based on a feature transposition technique, and/or using a diffusion image generation module, although implementations are not limited to these techniques.

In some implementations, methods can include the one or more processors generating a plurality of digital representations of a plurality of virtual clones having respective sets of predetermined characteristics including e.g. trace metal levels although embodiments are not limited thereto. In at least these implementations, methods can include determining a predicted effect on a PQA of the digital representation of varying one or more characteristics. PQAs can include any PQAs described earlier herein, including at least one of antibody stability, efficacy, safety, and potency.

FIG. 6A illustrates use of a virtual clone in a machine learning process 600. In the process 600, the digital twins (e.g., virtual clones that exhibit a predetermined set of characteristics) are generated to develop machine learning models 608 for predicting performance metrics and/or product quality metrics and/or to refine upstream process development techniques 610. By creating virtual clones, challenges associated with limited sets of historical training data can be overcome.

To this end, the computer system 102 may be configured to generate pools of virtual clones that have varying characteristics. More particularly, the characteristics may be varied in predefined manner to be able to test the impact of changes to particular characteristics (e.g., cell size, cell aggregation, and/or any of the other features described herein) on the performance metrics and/or product quality metrics. That is, by virtually varying characteristics of the virtual clone in a predefined manner, the training process for a machine learning model 608 is able to learn the relationships between virtual clone characteristics and performance metrics and/or product quality metrics. As a result, the predictive accuracy of relative performance (or even, in some embodiments, absolute performance) is improved. In one example, the virtual clones 602 are used to provide additional training data for training the classification model applied by the computer system 102 at stage 206 of the process 200.

In the illustrated example, the data 604 (e.g., the exported data generated via the method 500) is used as historical data from which the virtual clones 602 are generated. For example, image data associated with the virtual clones can be generated using feature transposition techniques and/or an image generation model (such as a diffusion model). The virtual clones 602 may then be input into a modeling platform 606 configured to perform in silico simulations of cell line development processes to simulate the growth of the virtual clones. It should be appreciated that the input parameters to the modeling platform 606 may vary from the feature set used as inputs to the ML model 608. Accordingly, when the computer system 102 varies the characteristics of the virtual clones, the computer system 102 may also vary these characteristics that are only used as inputs to the modeling platform 606.

FIG. 6B illustrates a composite pipeline 650 that combines the discrete components depicted in FIG. 6A. In the illustrated example, the digital twins (e.g., the virtual clones 602) can be applied for prediction of PQAs to determine antibody stability, efficacy, safety and potency. Process changes can be studied using the digital twins, rather than on physical clones. In one use case, the impact of increasing trace metal concentration can be studied using digital twins. In another use case, the digital twin can be used to capture scale impact on PQAs. In one example use case, batch medium modification can be studied using the digital twins.

Other attributes or effects of changing cell cultures can also be studied. For example, as shown in FIG. 7A-7C, digital twins can identify the PQAs most affected by nutrient spikes. In at least use cases, digital twins can be leveraged to run in silico process optimization, and run through various scenarios and possibilities for parameter changes, condition changes, etc. As shown in FIG. 7A, PQ1 is predicted to experience little detrimental impact from a nutrient spike, based on in silico experiments performed with digital twins and/or virtual clones 602. As shown in FIG. 7B, PQ2 is predicted to experience slightly more detrimental impact than PQ1, while the detrimental impact may remain below a threshold. As shown in FIG. 7C, PQ3 is predicted to experience detrimental impact.

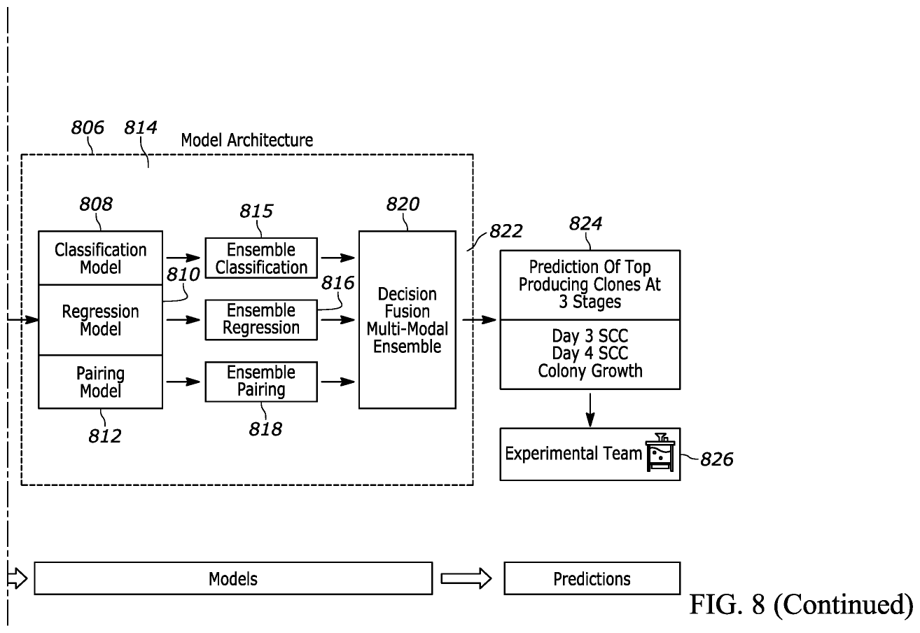

FIG. 8 illustrates a machine learning pipeline 800 for predicting top-producing clones in a cell line development process. The pipeline 800 depicts the flow of data from input sources through feature engineering, model processing, and prediction output stages. The development pipeline 800 may be implemented by the processing unit 120 of the computing system 102.

The pipeline 800 receives data inputs 802 from multiple data sources. For example, the inputs 802 may include image metadata (e.g., imaging assessment data stored in a cloud storage service and/or a distributed file system) generated by the cell clone generation and analysis system 110. As another example, the inputs 802 may include cell line development (CLD) process data generated by enterprise data systems (e.g., non-imaging assessment data). In some embodiments, the CLD process data may be manually compiled. As yet another example, the inputs 802 may include bioreactor data from a bioreactor system 150 (e.g., data from AMBR250 bioreactor runs).

As illustrated, the obtained data flows into an automatic data pipeline 803 that processes the data inputs 802. The data inputs may be recorded at different times throughout the CLD process, such as at a particular day or in response to detecting a development milestone (e.g., a colony growth milestone).

After receiving the data inputs 802, the data pipeline 803 proceeds to extract features of the data input 802 at a feature engineering stage 804. The feature engineering stage 804 may include a plurality of processing steps that generate high-quality, consistent features for model input. The extracted features may correspond to the input features for the respective feature spaces of the models of the model architecture 806. To generate the features, the feature engineering stage 804 may include respective feature extraction models that derive the features that are analyzed via the model architecture 806. For example, the feature extraction models may include image analysis models, mathematical models, and/or any other feature extraction model type known to those in the art. As described herein, because the particular models included the model architecture may be manually or automatically derived (e.g., to balance the predictive accuracy vs. time preferences), the particular feature extraction models may be correspondingly updated such that features not relevant to a model of the model architecture 806 are not generated.

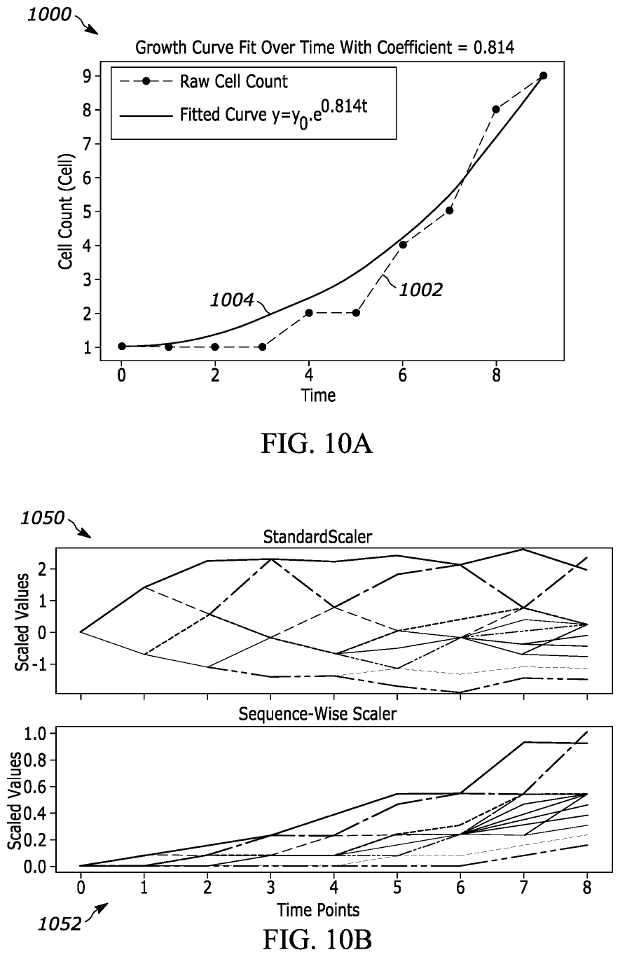

Different feature extraction models may be applied to different portions of the data inputs 802. For example, a first feature extraction model may extract features from process data and a second feature extraction model may extract features from image data. Similarly, different feature extraction models may be applied to portions of the data inputs 802 generated at or across different times in the CLD process. With respect to growth kinematics features, the feature engineering stage 804 may include a growth kinetics feature extraction model. As illustrated in FIG. 10A discussed later herein, the growth kinetics feature extraction model may fit a time series of cell count data in the input data 802 to an exponential growth model expressed as y=y₀×e^{rt}, where y represents cell count, y₀ represents initial cell count, r represents the growth rate constant, and t represents time. The computing system 102 may fit experimental cell count data to the exponential growth equation using nonlinear regression and output the fitted growth rate (r) and residuals (e.g., sum of squared residuals) as features. Accordingly, the growth rate constant may be used as an input feature that provides a quantitative, reproducible metric for comparing cell expansion rates across different clones.

As another example, an aggregation feature extraction model may generate aggregation features by applying a convolutional neural network (CNN) to pen images to classify each pixel into one of a plurality of categories, such as background, occupied by non-aggregated cells, and occupied by aggregated cells. An aggregation fraction may be calculated as the ratio of pixels classified as occupied by aggregated cells to the total number of pixels classified as occupied by cells. This fraction may then be used as an input feature to the model architecture 806 to provide an indication of cell aggregation that is informative of predicted cell line performance.

In some embodiments, the feature engineering stage 804 may include pre-processing the data inputs 802 prior to performing the feature extraction. In some embodiments, the pre-processing includes quality control filtering, in which the computing system 102 removes data from clones that fail to meet experimental criteria. Quality control criteria may include a single-cell origin requirement (e.g., each pen starts with exactly one cell to maintain monoclonality), a minimum cell count threshold (e.g., cell count of at least three at a predetermined timepoint before a secretion assay), a valid pool assignment requirement (e.g., each clone belongs to a defined pool identifier), and/or anomalous clones detected via an anomaly detection model.

In some embodiments, the pre-processing performed at the feature engineering stage 804 includes imputation techniques for filling in missing values in time-series assessment data. For example, the computing system 102 may apply spline imputation, which fits a smooth curve through available data points and estimates missing values based on the fitted curve. Spline imputation may preserve natural temporal patterns in cell behavior and may reduce abrupt or artificial jumps in the data, thereby providing more biologically meaningful imputed values than simpler methods such as filling with a mean value or a previous value. It should be appreciated that other imputation techniques are also envisioned.

In some embodiments, the pre-processing performed at the feature engineering stage 804 includes normalization techniques. The normalization techniques may be performed to normalize across the set of input data 802 such that the input data 802 for different candidate clones are more comparable and/or with respect to the training data such that the input data 802 exhibits characteristics aligned with expected data by the modal architecture 806. For example, the feature engineering stage 804 may perform sequence-wise normalization, as illustrated in FIG. 10B described later herein. As described earlier herein, the sequence-wise normalization scales measurements of a same feature across a plurality of timepoints as a single sequence, rather than normalizing each timepoint independently. This approach may preserve temporal patterns in the data and may improve data comparability across experiments.

Additionally or alternatively, the normalization techniques implemented by the feature engineering stage 804 include chip-wise normalization. To this end, due to the small size of cell imaging sensors, the physical image sensors used to image each pen may exhibit varying characteristics due to differences in manufacturing or calibration, differences caused by usage, or differences in operators or handling. Accordingly, the chip normalization techniques adjust the intensity or measurement scale for each well to account for chip-specific variations, for example, by capturing an empty-pen for each pen to generate a normalization factor that scales an intensity for each image sensor to a comparable baseline intensity. Chip-wise normalization may remove these non-biological biases so that differences in the data reflect biological differences rather than technical or positional biases.

In some embodiments, the feature engineering stage 804 includes performing one or more feature optimization techniques to reduce the set of features input to the model architecture 806. For example, the training process for the various models of the model architecture 806 may implement a dimension reduction algorithm, such a principal component analysis (PCA) or a latent discriminant analysis (LDA) to reduce the size of the feature space. Similarly, the training process may implement a feature selection algorithm, such as LASSO, to select only the most predictive features for the feature space. Accordingly, the feature engineering stage 804 may implement the feature reduction algorithm and/or output only the selected features for advancing the optimized feature set to the model architecture 806.

After generating the output features, the computing system 102 inputs the features from the feature engineering stage 804 into a model architecture 806 to generate one or more performance predictions. The model architecture 806 includes one or more base models, such as a classification model 808, a regression model 810, and a pairing model 812 trained to generate performance predictions in accordance with the techniques described elsewhere herein. It should be appreciated that in some embodiments, the base models 808, 810, 812 include multiple underlying base models 808, 810, 812 configured to predict a different performance indicator and/or accept a different set of input features (e.g., features characterizing cell line development at different times). Accordingly, the computing system 102 may provide the outputs from the base models to ensemble models 814 to generate ensemble predictions for each base model type. That is, an ensemble classification model 815 may generate ensemble predictions for classification models 808, an ensemble regression model 816 may generate ensemble predictions for regression models 810, and an ensemble pairing model 818 may generate ensemble predictions for pairing models 812. It should be appreciated that in embodiments where only a single base model of each type is implemented, the corresponding ensemble model 814 may be omitted from the model architecture 806 and the output routed directly to the fusion model 820.

The model architecture may then route the outputs of the ensemble models 820 The ensemble models 814 provide inputs to a decision fusion multi-modal ensemble 820, which combines the different modeling approaches as described earlier herein with reference to the decision fusion layer. The decision fusion multi-modal ensemble 820 generates a set of clones 824 to advance to a subsequent stage of the cell line development process. For example, the decision fusion multi-modal ensemble 820 may analyze the outputs of the ensemble models 814 (or base models 808, 810, 812) to identify which cell lines to advance. For example, the ensemble model 815 may associate each pen with confidence that the cell in the pen will exhibit the performance associated with the classification, the ensemble model 816 may associate each pen with a predict value for one or more performance metrics (and/or a composite metric derived therefrom), and the ensemble model 818 may output a ranked list of pens based on pair-wise comparison outcomes Accordingly, based on the sorted outputs of the ensemble models 814, the decision fusion multi-modal ensemble 820 may select the top performing pens from each list and/or or implement a regression model that combines the rankings into a single list.

As illustrated, in some embodiments, the selection of candidate cells may be an iterative process where, as additional input data 802 is compiled over time, the decision fusion multi-modal ensemble 820 outputs updated predictions such that the top performing pens not previously selected are added to the output set of clones 824 for advancement. For example, the decision fusion multi-modal ensemble 820 may output sets of top producing clones at three times: Day 3 SCC, Day 4 SCC, and colony growth.

The set of clones 824 may then be provided as an output of the computing system 102. In some embodiments, the output is provided via a display for an experimental team 826 to manually advance the selected clones. For example, the output may be a list of pen locations having the selected cells, a visual map of the selected cells, and so on. Similarly, the computing system 102 may update a virtual clone object corresponding to the analyzed clones to include an indication of the clone selection decision by the decision fusion multi-modal ensemble 820.

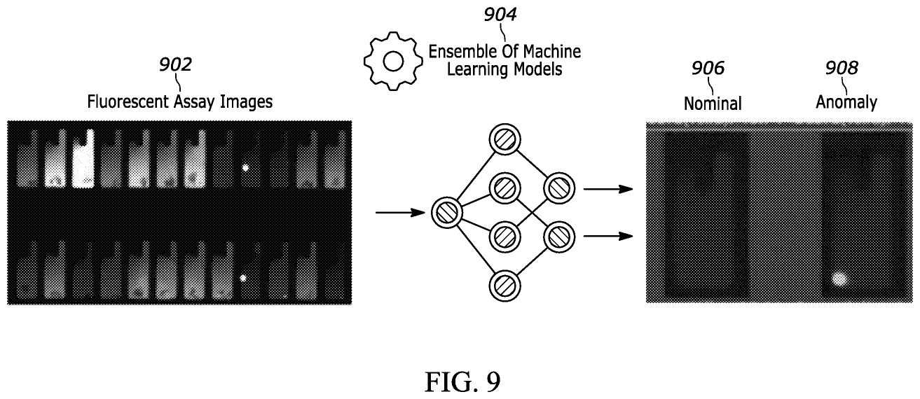

FIG. 9 illustrates anomaly detection in fluorescent assay images using machine learning. The anomaly detection techniques may be implemented to screen anomalous pens during the feature engineering stage 804. Inputs may include fluorescent assay images 902. Images 902 can include a grid of individual well images displaying varying fluorescence intensities. The computing system 102 processes the fluorescent assay images 902 using an ensemble of machine learning models 904, which may include convolutional neural networks (CNNs), support vector machines (SVMs), and autoencoders. The ensemble of machine learning models 904 collectively analyzes the fluorescent assay images 902 to identify anomalous cell phenotypes.

In some embodiments, the ensemble of machine learning models 904 includes a convolutional autoencoder that generates reconstruction errors serving as anomaly scores for cell phenotypes. The computing system 102 may train the autoencoder on images of clones exhibiting a desired phenotype (e.g., non-aggregated cells with approximately equal brightness). The autoencoder may generate higher reconstruction errors for images of clones exhibiting undesired phenotypes, thereby enabling automated identification of anomalous clones without requiring manual review by a subject matter expert.

The ensemble of machine learning models 904 generates classification outputs that categorize each analyzed image as either a nominal classification 906 or an anomaly classification 908. The nominal classification 906 indicates that the cell phenotype in the image is within expected parameters (e.g., all cells are round, cell borders are clearly visible, cells are approximately the same size and brightness, and cells are not merged together). The anomaly classification 908 indicates that the cell phenotype exhibits one or more anomalous characteristics, such as abnormal cell aggregation (e.g., cells merged together), abnormal cell brightness (e.g., one or more cells brighter than others), or abnormal cell morphology (e.g., cells that are not round or whose borders cannot be clearly distinguished).

In some embodiments, the classification results are displayed through an assay analyzer interface (not shown in FIG. 9), which provides a graphical user interface showing multiple culture columns with associated data fields including cell counts, viability metrics, and classification status for each analyzed well. The assay analyzer interface enables users to review the classification results and identify candidate clones that should be excluded from further analysis based on the anomaly detection performed by the ensemble of machine learning models 904.

FIG. 10A depicts a graph 1000 illustrating a growth curve fit over time. The graph plots cell count on the vertical axis against time on the horizontal axis. A raw cell count data series 1002 shows cell counts measured at successive timepoints, and a fitted curve 1004 follows an exponential growth model expressed as y=y₀×e^{rt}, where y represents cell count, y₀ represents initial cell count, r represents the growth rate constant, and t represents time. In the illustrated example, the fitted growth rate coefficient is 0.814 although embodiments are not limited thereto. The fitted exponential curve 1004 approximates the overall growth trend of the raw cell count data, demonstrating how the growth kinetics of cell colonies can be characterized by fitting observed cell count measurements to an exponential growth model to derive growth rate coefficients. As described earlier herein with reference to FIG. 8, the computing system 102 may use the growth rate coefficient and residuals from the curve fitting as features in the feature engineering stage 804.

FIG. 10B depicts a comparison of two data scaling approaches applied to time-series data across multiple timepoints. An upper panel 1050 illustrates results from a standard normalization technique, in which each timepoint column is scaled independently. Multiple data sequences exhibit varying trajectories, with some sequences trending upward, others trending downward, and some remaining relatively stable across the timepoints. A lower panel1 1052 illustrates results from a sequence-wise normalization technique, in which each data sequence is scaled as a single unit across all timepoints. The data sequences in the lower panel generally trend upward across the timepoints, preserving the temporal growth pattern. The comparison demonstrates that standard normalization may distort temporal patterns by scaling each timepoint independently, while sequence-wise normalization may preserve the biological trajectory by scaling measurements of a same feature across a plurality of timepoints as a single sequence, as described earlier herein with reference to FIG. 8.

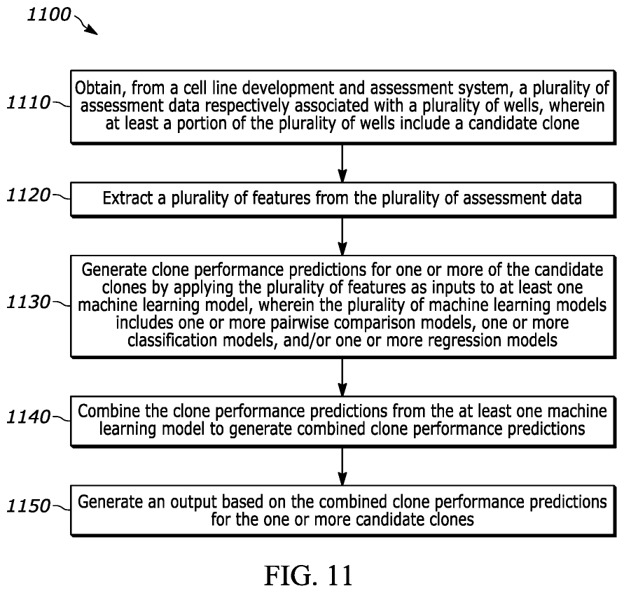

FIG. 11 illustrates a flowchart for a method 1100 of cell clone assessment. The method 1100 may be implemented by the computing system 102 described earlier herein with reference to FIG. 2.

The method 1100 begins with operation 1110, with the computing system 102 obtaining, from a cell line development and assessment system (e.g., the cell line development and analysis system 110), a plurality of assessment data respectively associated with a plurality of wells. At least a portion of the plurality of wells may include a candidate clone. In some embodiments, the plurality of assessment data is obtained from the cell line development and assessment system during a first stage of a cell line development pipeline, and the clone performance predictions generated by the method 1100 are indicative of predicted performance at a bioreactor system (e.g., the bioreactor system 150) during a subsequent stage of the cell line development pipeline. In some embodiments, the cell line development and assessment system comprises a high-throughput microfluidic screening system configured to screen candidate clones, and the bioreactor system is configured to evaluate candidate clones advanced from the cell line development and assessment system. In some embodiments, the two or more machine learning models used in the method 1100 are trained using historical training data that includes historical assessment data from cell line development and assessment systems and corresponding historical performance data from bioreactor systems as ground truth data. The ground truth data may comprise one or more of titer values, viable cell density values, specific productivity values, and purity values measured at the bioreactor system. The historical training data may be associated with a plurality of historical programs associated with a plurality of molecule modalities and may include historical performance data obtained from a plurality of bioreactor runs for each of a plurality of historical clones.

The method 1100 may continue with operation 1120, with the computing system 102 extracting a plurality of features from the plurality of assessment data. The plurality of features may comprise one or more of time-dependent morphological features measured at a plurality of timepoints (including one or more of cell size features, cell shape features, and cell morphology features), growth kinetics features derived from fitting cell count data to a growth model, secretion features derived from fluorescent assay data, and aggregation features derived from pixel classification of cell images. In some embodiments, extracting the plurality of features comprises normalizing an intensity of image data using a rolling median calculated from empty wells within a chip segment, normalizing the plurality of assessment data on a per-molecule basis across different molecule modalities, and/or applying a sequence-wise normalization to time-series features, wherein the sequence-wise normalization scales measurements of a same feature across a plurality of timepoints as a single sequence. In some embodiments, prior to generating clone performance predictions, the computing system 102 applies an anomaly detection model to image data associated with the plurality of wells and excludes candidate clones identified as anomalous from the clone performance predictions. The anomaly detection model may comprise an ensemble of anomaly detection models including one or more of a support vector machine, an autoencoder, and a convolutional neural network. In some embodiments, the anomaly detection model is trained using a set of manually labeled images identifying anomalous cell phenotypes, and the anomalous cell phenotypes include one or more of cell aggregation, abnormal cell brightness, and abnormal cell morphology.

Following feature extraction, the method 1100 may continue with operation 1130, with the computing system 102 generating clone performance predictions for one or more of the candidate clones by applying the plurality of features as inputs to two or more machine learning models. The two or more machine learning models may include one or more pairwise comparison models, one or more classification models, and/or one or more regression models. In some embodiments, the two or more machine learning models comprise a pairwise comparison model, and generating clone performance predictions comprises selecting a pair of candidate clones (e.g., candidate clones having a common parent cell line), predicting which candidate clone of the pair has a superior clone performance metric, and iteratively selecting pairs of candidate clones and aggregating predictions to generate a ranking of candidate clones. The output may comprise an ordered list of candidate clones ranked based on the aggregated predictions. In some embodiments, the two or more machine learning models comprise a classification model, and the output comprises a classification of one or more candidate clones with respect to a clone performance metric. The classification may comprise a binary classification indicating whether each of the one or more candidate clones is predicted to satisfy a condition associated with the clone performance metric. In some embodiments, the two or more machine learning models comprise a regression model, and the output comprises a predicted value for a clone performance metric for one or more candidate clones. The predicted value may comprise one or more of a predicted titer value, a predicted viable cell density value, a predicted specific productivity value, and a predicted purity value, and the plurality of features may be captured at a predetermined time during a cell line screening process.

The method 1100 may continue with operation 1140, with the computing system 102 combining the clone performance predictions from the two or more machine learning models to generate combined clone performance predictions. In some embodiments, combining the clone performance predictions comprises inputting the outputs from the two or more machine learning models into a fusion model to generate the combined clone performance predictions. The fusion model may select highest performing candidate clones with respect to the two or more machine learning models. In some embodiments, the computing system 102 presents a user interface configured to obtain prediction configuration information including one or more of a parent cell line, a molecule modality, a target clone performance metric, and a prediction timing, and selects the two or more machine learning models from a model database based on the prediction configuration information. The model database may maintain a plurality of models of a same type, each trained using assessment data captured at a respective timepoint during a cell line screening stage. The model database may also maintain a plurality of models configured to predict different target clone performance metrics.

The method 1100 may continue with operation 1150, with the computing system 102 generating an output based on the combined clone performance predictions for the one or more candidate clones. In some embodiments, the method 1100 further comprises obtaining performance data associated with the clone performance for the one or more candidate clones during a subsequent stage of a development pipeline, and updating the two or more machine learning models using the performance data as ground truth data.

In some embodiments, the method 1100 further includes selecting a set of candidate clones for advancement to a subsequent clone screening stage based on the output. In these embodiments, the method may include displaying, via a display, the selected set of candidate clones as one or more of a list of well identifiers for the wells housing the candidate clones and a set of indications on a map of wells. In some embodiments, the subsequent stage may be a stage of a cell line development pipeline for production of a biologic drug product. In some further embodiments, the method may include generating a master cell bank from the candidate clones selected based on the output.

Although the systems, methods, devices, and components thereof, have been described in terms of exemplary embodiments, they are not limited thereto. The detailed description is to be construed as exemplary only and does not describe every possible embodiment of the invention because describing every possible embodiment would be impractical, if not impossible. Numerous alternative embodiments could be implemented, using either current technology or technology developed after the filing date of this patent that would still fall within the scope of the claims defining the invention.

Those skilled in the art will recognize that a wide variety of modifications, alterations, and combinations can be made with respect to the above-described embodiments without departing from the scope of the invention, and that such modifications, alterations, and combinations are to be viewed as being within the ambit of the inventive concept.

Additional details encompassed by the instant techniques are described in the attached appendix.

1.-39. (canceled)

40. A method of cell clone assessment, the method comprising:
obtaining, by one or more processors and from a cell line development and assessment system, a plurality of assessment data respectively associated with a plurality of wells, wherein at least a portion of the plurality of wells include a candidate clone;
extracting, by the one or more processors, a plurality of features from the plurality of assessment data;
generating, by the one or more processors, clone performance predictions for one or more of the candidate clones by applying the plurality of features as inputs to two or more machine learning models, wherein the two or more machine learning models includes one or more pairwise comparison models, one or more classification models, and/or one or more regression models;
combining, by the one or more processors, the clone performance predictions from the two or more machine learning models to generate combined clone performance predictions; and
generating, by the one or more processors, an output based on the combined clone performance predictions for the one or more candidate clones.

41. The method of claim 40, wherein:
the plurality of assessment data is obtained from the cell line development and assessment system during a first stage of a cell line development pipeline, and
the clone performance predictions are indicative of predicted performance at a bioreactor system during a subsequent stage of the cell line development pipeline.

42. The method of claim 41, wherein:
the cell line development and assessment system comprises a high-throughput microfluidic screening system configured to screen candidate clones, and
the bioreactor system is configured to evaluate candidate clones advanced from the cell line development and assessment system.

43. The method of claim 41, wherein:
the two or more machine learning models are trained using historical training data, and
the historical training data includes historical assessment data from cell line development and assessment systems and corresponding historical performance data from bioreactor systems as ground truth data, and
the ground truth data comprises one or more of titer values, viable cell density values, specific productivity values, and purity values measured at the bioreactor system.

44.-46. (canceled)

47. The method of claim 40, further comprising:
obtaining performance data associated with clone performance for the one or more candidate clones during a subsequent stage of a development pipeline; and
updating the two or more machine learning models using the performance data as ground truth data.

48. The method of claim 40, wherein:
the two or more machine learning models comprise a pairwise comparison model, and
generating clone performance predictions comprises:
selecting a pair of candidate clones;
predicting which candidate clone of the pair has a superior clone performance metric; and
iteratively selecting pairs of candidate clones and aggregating predictions to generate a ranking of candidate clones.

49. (canceled)

50. The method of claim 48, wherein:
the output comprises an ordered list of candidate clones ranked based on the aggregated predictions.

51. The method of claim 40, wherein:
the two or more machine learning models comprise a classification model, and
the output comprises a classification of one or more candidate clones indicating whether each of the one or more candidate clones is predicted to satisfy a condition associated with a clone performance metric.

52. (canceled)

53. The method of claim 40, wherein:
the two or more machine learning models comprise a regression model, and
the output comprises a predicted value for a clone performance metric for one or more candidate clones.

54.-55. (canceled)

56. The method of claim 40, wherein combining the clone performance predictions comprises:
inputting the outputs from the two or more machine learning models into a fusion model to generate the clone performance predictions.

57. The method of claim 56, wherein the fusion model is configured to select highest performing candidate clones with respect to the two or more machine learning models.

58. The method of claim 40, further comprising:
presenting, by the one or more processors, a user interface configured to obtain prediction configuration information including one or more of a parent cell line, a molecule modality, a target clone performance metric, and a prediction timing; and
selecting, by the one or more processors, the two or more machine learning models from a model database based on the prediction configuration information.

59. The method of claim 58, wherein:
the model database maintains (i) a plurality of models of a same type, each trained using assessment data captured at a respective timepoint during a cell line screening stage and/or (ii) a plurality of models configured to predict different target clone performance metrics.

60. (canceled)

61. The method of claim 40, wherein extracting the plurality of features comprises:
normalizing an intensity of image data using a rolling median calculated from empty wells within a chip segment.

62. The method of claim 40, wherein extracting the plurality of features comprises:
normalizing the plurality of assessment data on a per-molecule basis across different molecule modalities.

63. The method of claim 40, wherein extracting the plurality of features comprises:
applying a sequence-wise normalization to time-series features, wherein the sequence-wise normalization scales measurements of a same feature across a plurality of timepoints as a single sequence.

64. The method of claim 40, further comprising:
prior to generating clone performance predictions, applying an anomaly detection model to image data associated with the plurality of wells; and
excluding candidate clones identified as anomalous from the clone performance predictions.

65. The method of claim 64, wherein:
the anomaly detection model comprises an ensemble of anomaly detection models including one or more of a support vector machine, an autoencoder, and a convolutional neural network.

66. (canceled)

67. The method of claim 40, further comprising:
based on the output, selecting, by the one or more processors, a set of candidate clones for advancement to a subsequent clone screening stage.

68. The method of claim 67, further comprising:
displaying, via a display, the selected set of candidate clones as one or more of a list of well identifiers for the wells housing the candidate clones and a set of indications on a map of wells.

69.-70. (canceled)

71. A system comprising:
memory storing non-transitory computer-executable instructions; and
one or more processors coupled to the memory and configured to execute the instructions to:
obtain, from a cell line development and assessment system, a plurality of assessment data respectively associated with a plurality of wells, wherein at least a portion of the plurality of wells include a candidate clone;
extract a plurality of features from the plurality of assessment data;
generate clone performance predictions for one or more of the candidate clones by applying the plurality of features as inputs to two or more machine learning models, wherein the two or more machine learning models includes one or more pairwise comparison models, one or more classification models, and/or one or more regression models;
combine the clone performance predictions from the two or more machine learning models to generate combined clone performance predictions; and
generate an output based on the combined clone performance predictions for the one or more candidate clones.
